SEQUENCE LISTING (8 sequences)

SEQ ID NO: 1 - DNA, 51 bp, Artificial
  synthetic primer
ggggacaagtttgtacaaaaaagcaggctatgacccctcactcgattgacc

SEQ ID NO: 2 - DNA, 52 bp, Artificial
  synthetic primer
ggggaccactttgtacaagaaagctgggttcaccagtttcggcacttcttgc

SEQ ID NO: 3 - DNA, 49 bp, Artificial
  synthetic primer
ggggacaagtttgtacaaaaaagcaggctatgctcggcgctccctctcc

SEQ ID NO: 4 - DNA, 54 bp, Artificial
  synthetic primer
ggggaccactttgtacaagaaagctgggttcatgtctgcttctccaaaaacacc

SEQ ID NO: 5 - DNA, 51 bp, Artificial
  synthetic primer
ggggacaagtttgtacaaaaaagcaggctatgtcgcccagtgctgcagttc

SEQ ID NO: 6 - DNA, 51 bp, Artificial
  synthetic primer
ggggaccactttgtacaagaaagctgggtctagtgagtccttttcaggcgc

SEQ ID NO: 7 - DNA, 54 bp, Artificial
  synthetic primer
ggggacaagtttgtacaaaaaagcaggctatgatgatgctctcaaagagtctcc

SEQ ID NO: 8 - DNA, 55 bp, Artificial
  synthetic primer
ggggaccactttgtacaagaaagctgggttcatgtattcaggcattgcgagtacc

(19) United States
(12) Patent Application Publication
(10) Pub. No.: US 2010/0137184 A1
(43) Pub. Date: Jun. 3, 2010

(54) Cleaning Compositions Comprising Alpha-Galactosidase

(76) Inventors: Hugh C. McDonald, Carlsbad, CA (US); Ayrookaran J. Poulose, Belmont, CA (US)

Correspondence Address: DANISCO US INC.;ATTENTION: LEGAL DEPARTMENT, 925 PAGE MILL ROAD, PALO ALTO, CA, 94304, US

(21) Appl. No.: 12/528,986

(22) PCT Filed: Feb. 26, 2008
(86) PCT No.: PCT/US08/02473

§ 371 Date: Jan. 13, 2010

Related U.S. Application Data
(60) Provisional application No. 60/904,141, filed on Feb. 28, 2007.

(51) Int. Cl. C11D 7/42; C12N 9/40

(52) U.S. Cl. 510/218; 510/392; 510/320; 435/208

(57) ABSTRACT

The present invention provides cleaning compositions comprising an isolated alpha-galactosidase enzyme. In some particularly preferred embodiments, the isolated alpha-galactosidase enzyme comprises an amino acid sequence that is related to an alpha-galactosidase from Trichoderma reesei. The present invention also provides methods for using the alpha-galactosidase in cleaning applications.

### FIELD OF THE INVENTION

The present invention provides cleaning compositions comprising an isolated alpha-galactosidase enzyme. In some particularly preferred embodiments, the isolated alpha-galactosidase enzyme comprises an amino acid sequence that is related to an alpha-galactosidase from Trichoderma reesei. The present invention also provides methods for using the alpha-galactosidase in cleaning applications.

### BACKGROUND OF THE INVENTION

Detergent and other cleaning compositions often include a complex combination of active ingredients. For example, certain cleaning products contain a surfactant system, enzymes for cleaning, bleaching agents, builders, suds suppressors, soil-suspending agents, soil-release agents, optical brighteners, softening agents, dispersants, dye transfer inhibition compounds, abrasives, bactericides, and perfumes. Despite the complexity of current detergents, there are many stains that are difficult to remove.

### SUMMARY OF THE INVENTION

The present invention provides cleaning compositions comprising an isolated alpha-galactosidase enzyme. In some particularly preferred embodiments, the isolated alpha-galactosidase enzyme comprises an amino acid sequence that is related to an alpha-galactosidase from Trichoderma reesei. The present invention also provides methods for using the same alpha-galactosidase in cleaning applications. In some embodiments, the cleaning composition further comprises at least one surfactant. In some preferred embodiments, the cleaning compositions have a working pH that is at least about pH 5.0. The present invention also provides methods for cleaning objects using the cleaning compositions of the present invention.

In some embodiments, the alpha-galactosidase enzyme has an amino acid sequence that is at least about 70%, at least about 80%, at least about 90%, at least about 95%, or at least about 98% identical to an alpha-galactosidase of Trichoderma reesei. In some other embodiments, the alpha-galactosidase enzyme is immunologically cross-reactive with the alpha-galactosidase of Trichoderma reesei.

In some embodiments, the cleaning compositions of the present invention are solids (e.g., a powder or a tablet), while in other embodiments, they are liquids, gels, foams, or other forms. In some preferred embodiments, the cleaning compositions are formulated as laundry detergents, dishwashing detergents, or laundry additives. So some preferred embodiments, the cleaning compositions further comprise at least one additional enzyme, including but not limited to enzymes such as a hemicellulases, mannanases, pectinases, or xylanases, useful for the degradation of non-starch food polysaccharides. In some yet further embodiments, the cleaning compositions further comprise at least one additional enzyme, including but not limited to enzymes such as proteases, amylases, cellulases, lipases, cutinases, or oxido-reductases, for the degradation of other stain components. Indeed, suitable enzymes that find use in combination with the alpha-galactosidase of the present invention include, but are not limited to hemicellulases, peroxidases, proteases, cellulases, xylanases, lipases, phospholipases, esterases, cutinases, pectinases, keratinases, reductases, oxidases, phenoloxidases, lipoxygenases, ligninases, pullulanases, tannases, pentosanases, malanases, β-glucanases, arabinosidases, hyaluronidase, chondroitinase, laccase, and amylases, or mixtures thereof. In some embodiments, a combination of enzymes (i.e., a “cocktail”) comprising conventional applicable enzymes like protease, lipase, cutinase and/or cellulase in conjunction with alpha-galactosidase is used.

The present invention also provides methods for cleaning, including the steps of contacting the isolated alpha-galactosidase enzyme with an object (e.g., a fabric or a dishware) under conditions suitable for activity of the alpha-galactosidase enzyme, to clean the object. In some embodiments, the alpha-galactosidase enzyme is contacted the object at a pH that is greater than about pH 5 (e.g., a pH in the range of about pH 5 to about pH 6.5, about pH 6.5 to about pH 7.5, about pH 7.5 to about pH 8.5, about pH 9.5 to about pH 10.5, or about pH 10.5 to about pH 11.5).

In some embodiments, the object is a soiled object (e.g., an object stained by a foodstuff) containing a non-starch food polysaccharide (e.g., a galactomannan gum such as guar gum or lima bean gum). Such foodstuffs include, but are not limited to salad dressings, ice cream, milkshakes, mousses, salad creams, and chocolate cream.

In some preferred embodiments, the cleaning compositions of the present invention remove stains from objects more effectively than equivalent cleaning compositions that do not contain alpha-galactosidase.

### DESCRIPTION OF THE DRAWINGS

Certain aspects of the following detailed description are best understood when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 shows a map of the pTrex3g vector.

FIG. 2 shows an SDS PAGE gel and two graphs showing the results of analysis of the AGL1 enzyme.

FIG. 3 shows an SDS PAGE gel and two graphs showing the results of analysis of the AGL2 enzyme.

FIG. 4 shows an SDS PAGE gel and two graphs showing the results of analysis of the AGL3 enzyme.

FIG. 5 is a graph showing the cleaning activity of beta-mannanase (NSP-20), AGL1 (NSP-6), AGL2 (NSP-8) and AGL3 (NSP-9) on chocolate cream stains.

FIG. 6 is a graph showing the cleaning activity of beta-mannanase (NSP-20) and AGL1 (NSP-6) on salad dressing stains.

FIG. 7 is a graph showing the cleaning activity of AGL2 (NSP-8) on guar pigment stains.

FIG. 8 is a graph showing the cleaning activity of beta-mannanase (NSP-20) and AGL2 (NSP-8) on chocolate ice cream stains.

FIG. 9 is a graph showing the cleaning activity of beta-mannanase (NSP-20), AGL1 (NSP-6), AGL2 (NSP-8) and AGL3 (NSP-9) on guar pigment stains in WFK automatic dishwasher detergent (ADW) and AATCC laundry detergent.

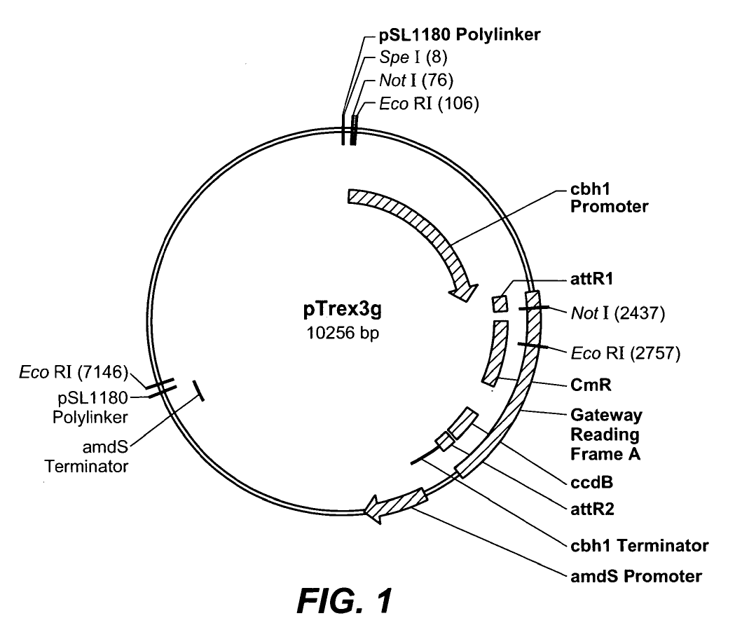

### DESCRIPTION OF THE INVENTION

The present invention provides cleaning compositions comprising an isolated alpha-galactosidase enzyme. In some particularly preferred embodiments, the isolated alpha-galactosidase enzyme comprises an amino acid sequence that is related to an alpha-galactosidase from Trichoderma reesei. The present invention also provides methods for using the alpha-galactosidase in cleaning applications.

Unless otherwise indicated, the practice of the present invention involves conventional techniques commonly used in molecular biology, microbiology, and recombinant DNA, which are within the skill of the art. Such techniques are known to those of skill in the art and are described in numerous texts and reference works well-known to those skilled in the art. All patents, patent applications, articles and publications mentioned herein, both supra and infra, are hereby expressly incorporated herein by reference. Unless defined otherwise herein, all technical and scientific terms used herein have the same meaning as commonly understood by one of ordinary skill in the art to which this invention pertains. Although any methods and materials similar or equivalent to those described herein find use in the practice of the present invention, the preferred methods and materials are described herein. Accordingly, the terms defined immediately below are more fully described by reference to the Specification as a whole.

Also, as used herein, the singular “a,” “an,” and “the” includes the plural reference unless the context clearly indicates otherwise. Numeric ranges are inclusive of the numbers defining the range. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Furthermore, the headings provided herein are not limitations of the various aspects or embodiments of the invention, which can be had by reference to the specification as a whole. Accordingly, the terms defined immediately below are more fully defined by reference to the specification as a whole. Nonetheless, in order to facilitate understanding of the invention, a number of terms are defined below.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All documents cited are, in relevant part, incorporated herein by reference; the citation of any document is not to be construed as an admission that it is prior art with respect to the present invention.

The term “recombinant” refers to a polynucleotide or polypeptide that does not naturally occur in a host cell. A recombinant molecule may contain two or more naturally-occurring sequences that are linked together in a way that does not occur naturally. A recombinant cell contains a recombinant polynucleotide or polypeptide.

The term “heterologous” refers to elements that are not normally associated with each other. For example, if a host cell produces a heterologous protein, that protein that is not normally produced in that host cell. Likewise, a promoter that is operably linked to a heterologous coding sequence is a promoter that is operably linked to a coding sequence that it is not usually operably linked to in a wild-type host cell. The term “homologous,” with reference to a polynucleotide or protein, refers to a polynucleotide or protein that occurs naturally in a host cell.

The terms “protein” and “polypeptide” are used interchangeably herein.

A “signal sequence” is a sequence of amino acids present at the N-terminal portion of a protein which facilitates the secretion of the mature form of the protein outside the cell. The definition of a signal sequence is a functional one. The mature form of the extracellular protein lacks the signal sequence which is cleaved off during the secretion process.

A “coding sequence” is a DNA segment that encodes a polypeptide.

The term “nucleic acid” encompasses DNA, RNA, single stranded or double stranded and chemical modifications thereof. The terms “nucleic acid” and “polynucleotide” are be used interchangeably herein.

A “vector” refers to a polynucleotide designed to introduce nucleic acids into one or more host cells. Vectors can autonomously replicated in different host cells and include cloning vectors, expression vectors, shuttle vectors, plasmids, phage particles, cassettes and the like.

An “expression vector” as used herein means a DNA construct comprising a protein-coding region that is operably linked to a suitable control sequence capable of effecting expression of the protein in a suitable host cell. Such control sequences may include a promoter to effect transcription, an optional operator sequence to control transcription to produce mRNA, a sequence encoding suitable ribosome binding sites on the mRNA, and enhancers and sequences which control termination of transcription and translation.

A “promoter” is a regulatory sequence that initiates transcription of a downstream nucleic acid.

The term “operably linked” refers to an arrangement of elements that allows them to be functionally related. For example, a promoter is operably linked to a coding sequence if it controls the transcription of the sequence.

The term “selective marker” refers to a protein capable of expression in a host that allows for ease of selection of those hosts containing an introduced nucleic acid or vector. Examples of selectable markers include but are not limited to antimicrobials (e.g., hygromycin, bleomycin, or chloramphenicol) and/or genes that confer a metabolic advantage, such as a nutritional advantage on the host cell.

The term “derived” encompasses the terms “originated from,” “obtained,” “obtainable from,” and “isolated from”.

A “non-pathogenic” organism is an organism that is not pathogenic to humans.

The terms “recovered,” “isolated,” and “separated” as used herein refer to a protein, cell, nucleic acid or amino acid that is removed from at least one component with which it is naturally associated.

As used herein, the terms “transformed,” “stably transformed,” and “transgenic” used in reference to a cell means the cell has a non-native (e.g., heterologous) nucleic acid sequence integrated into its genome or as an episomal plasmid that is maintained through multiple generations.

As used herein, the term “expression” refers to the process by which a polypeptide is produced based on the nucleic acid sequence of a gene. The process includes both transcription and translation.

The term “introduced” in the context of inserting a nucleic acid sequence into a cell, means “transfection,” “transformation,” or “transduction” and includes reference to the incorporation of a nucleic acid sequence into a eukaryotic or prokaryotic cell wherein the nucleic acid sequence may be incorporated into the genome of the cell (e.g., chromosome, plasmid, plastid, or mitochondrial DNA), converted into an autonomous replicon, or transiently expressed (e.g., transfected mRNA).

The term “hybridization” refers to the process by which a strand of nucleic acid joins with a complementary strand through base pairing as known in the art. A nucleic acid is considered to be “Selectively hybridizable” to a reference nucleic acid sequence if the two sequences specifically hybridize to one another under moderate to high stringency hybridization and wash conditions. Moderate and high stringency hybridization conditions are well-known to those skilled in the art. One example of high stringency conditions include hybridization at about 42 C in 50% formamide, 5×SSC, 5×Denhardt's solution, 0.5% SDS and 100 ug/ml denatured carrier DNA followed by washing two times in 2×SSC and 0.5% SDS at room temperature and two additional times in 0.1×SSC and 0.5% SDS at 42 C.

As used herein, “cleaning composition” and “cleaning formulation” refer to a composition that finds use in the removal of undesired compounds (e.g., a stain) from items to be cleaned, such as fabric, dishes, contact lenses, other solid substrates, hair (shampoos), skin (soaps and creams), teeth (mouthwashes, toothpastes), etc. It is not intended that the present invention be limited to any particular formulation, as the terms encompass any materials/compounds selected for the particular type of cleaning composition desired and the form of the product (e.g., liquid, gel, granule, or spray composition), as long as the composition is compatible with the subject enzyme in the composition. The specific selection of cleaning composition materials is readily made by considering the surface, item or fabric to be cleaned, and the desired form of the composition for the cleaning conditions during use.

It is intended that the terms include, but are not limited to detergent compositions (e.g., liquid and/or solid laundry detergents and fine fabric detergents; hard surface cleaning formulations, such as for glass, wood, ceramic and metal counter tops and windows; carpet cleaners; oven cleaners; fabric fresheners; fabric softeners; and textile and laundry pre-spotters, as well as dish detergents).

Indeed, the term “cleaning composition” as used herein includes, unless otherwise indicated, granular, tablet or powder-form all-purpose or heavy-duty washing agents, especially cleaning detergents; liquid, gel or paste-form all-purpose washing agents, especially heavy-duty liquid (HDL) types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-washes, cleaning bars, mouthwashes, denture cleaners, car or carpet shampoos, bathroom cleaners; hair shampoos and hair-rinses; shower gels and foam baths and metal cleaners; as well as cleaning auxiliaries such as bleach additives and “stain-stick,” pre-treatment or laundry additives.

As used herein, the terms “detergent composition” and “detergent formulation” are used in reference to compositions that formulated for use in a wash medium for the cleaning of soiled objects. In particular embodiments, the term is used in reference to laundering fabrics and/or garments (e.g., “laundry detergents”). In alternative embodiments, the term refers to other detergents, such as those used to clean dishes, cutlery, etc. (e.g., “dishwashing detergents”). It is not intended that the present invention be limited to any particular detergent formulation or composition. Indeed, in some embodiments, the detergent compositions contain surfactants, transferase(s), hydrolytic enzymes, oxido reductases, builders, bleaching agents, bleach activators, bluing agents and fluorescent dyes, caking inhibitors, masking agents, enzyme activators, antioxidants, and/or solubilizers, etc., in addition to alpha-galactosidase.

As used herein, “enhanced performance” in a cleaning composition is defined as increasing cleaning (e.g., removal and/or decolorization) of stains. In some preferred embodiments, the stains are galactomannan-related stains (e.g., chocolate cream, salad dressings, guar, etc.), as determined by usual evaluation after a standard wash cycle.

As used herein the term “hard surface cleaning composition,” refers to detergent compositions for cleaning hard surfaces such as floors, walls, tile, bath and kitchen fixtures, and the like. Such compositions are provided in any form, including but not limited to solids, liquids, emulsions, etc.

As used herein, “dishwashing composition” refers to all suitable forms for compositions for cleaning dishes, including but not limited to granules, gels, emulsions, and liquids.

As used herein, “fabric cleaning composition” refers to all forms of detergent compositions for cleaning fabrics, including but not limited to, granules, liquids, gels, emulsions, and bars.

As used herein, “textile” refers to woven fabrics, as well as staple fibers and filaments suitable for conversion to or use as yarns, woven, knit, and non-woven fabrics. The term encompasses yarns made from natural, as well as synthetic (e.g., manufactured) fibers.

As used herein, “textile materials” is a general term for fibers, yarn intermediates, yarn, fabrics, and products made from fabrics (e.g., garments and other articles).

As used herein, “fabric” encompasses any textile material. Thus, it is intended that the term encompass garments, as well as fabrics, yarns, fibers, non-woven materials, natural materials, synthetic materials, and any other textile material.

As used herein, “effective amount of alpha-galactosidase” refers to the quantity of alpha-galactosidase enzyme necessary to achieve the enzymatic activity required in the specific application (e.g., cleaning composition, etc.). Such effective amounts are readily ascertained by one of ordinary skill in the art and are based on many factors, such as the particular enzyme variant used, the cleaning application, the specific composition of the cleaning composition, and whether a liquid or dry (e.g., granular, bar) composition is required, and the like.

The terms “α-galactosidase” and alpha-galactosidase refer to an enzyme that hydrolyses terminal, non-reducing alpha-D-galactose residues in alpha-D-galactosides, including galactose oligosaccharides and galactomannans. The alpha-galactosidase described herein has an activity described as EC 3.2.1.22, according to IUBMB enzyme nomenclature. The systematic name for the alpha-galactosidase described herein is alpha-D-galactoside galactohydrolase.

The term “soiled object” refers to an object (e.g., a fabric or dish), that is soiled, (e.g., stained) with a second composition. Encompassed by the term “soiled object” are dirty fabrics, such as dirty clothing, linens, and fabrics that are stained with foodstuffs containing non-starch food polysaccharides. In certain embodiments, the stain has a visible color.

The term “non-starch food polysaccharide” refers to a non-starch polysaccharide that is employed as a filler, thickener, stabilizer or binder of free water in many foodstuffs (e.g., sauces, creams, dairy products, ice creams, mousses, milkshakes and salad dressings). Guar gum, an edible thickening agent extracted from the leguminous guar bean shrub, and locust bean gum, which is extracted from the seeds of the carob tree, are examples of non-starch food polysaccharide.

The term “non-starch food polysaccharide degrading enzyme” refers to an enzyme that degrades non-starch food polysaccharides. Exemplary enzymes include, but are not limited to, hemicellulase, mannanase, pectinase, xylanase, beta-galactosidase and alpha-galactosidase.

The term “galactomannan gum” refers to a plant-derived polysaccharide that is composed of polymers containing galactose and mannose residues. Guar gum, tara gum, fenugreek gum, and bean gum are types of galactomannan gums.

The term “working pH” refers to the pH of a detergent during its use. For example, the working pH of a laundry detergent is the pH of the detergent when it is used to wash fabrics in a washing machine or during hand washing. Likewise, the working pH of a dishwashing detergent is the pH of that detergent as it is being used in a dishwasher or in hand dishwashing. In some embodiments, detergents that are in concentrated or solid form are diluted or dissolved before the pH of that detergent is at its working pH.

The term “working concentration” refers to the concentration of an enzyme in a detergent during use. For example, the working concentration of an enzyme in a laundry detergent is the concentration of that enzyme when the laundry detergent is used to wash fabrics in a washing machine or during hand washing Likewise, the working concentration of an enzyme in a dishwashing detergent is the concentration of that enzyme in the detergent as it is being used in a dishwasher or during hand washing. In some embodiments, detergents that are in concentrated or solid form are diluted or dissolved before the concentration of an enzyme in a detergent is at its working concentration.

The present invention provides cleaning compositions comprising an isolated alpha-galactosidase enzyme comprising an amino acid sequence that is related to (e.g., at least about 90% identical) to an alpha-galactosidase of Trichoderma reesei. In some embodiments, the cleaning composition comprises at least one surfactant. In some embodiments, the cleaning composition has a working pH that is at least about pH 5. The present invention also provides methods for cleaning objects that utilize the cleaning compositions provided herein.

Before the exemplary embodiments are described in more detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Although any methods and materials similar or equivalent to those described herein can be used in the practice or testing of the present invention, exemplary and preferred methods and materials are now described. All publications mentioned herein are incorporated herein by reference to disclose and describe the methods and/or materials in connection with which the publications are cited.

### Alpha-Galactosidase Enzyme

As noted above, the present invention provides cleaning compositions comprising an alpha-galactosidase enzyme. In some embodiments, the alpha-galactosidase enzyme has an amino acid sequence that is at least about 70%, at least about 80%, at least about 85%, at least about 90%, at least about 91%, at least about 92%, at least about 93%, at least about 94%, at least about 95%, at least about 96%, at least about 97%, at least about 98%, at least about 99% or about 100% identical to the amino acid sequence of a wild-type Trichoderma reesei alpha-galactosidase. The amino acid sequences for three examples of such enzymes are known in the art (See, Margolles-Clark et al., Eur. J. Biochem., 240:104-11 [1996]). The nucleotide sequence of mRNA encoding the Trichoderma reesei alpha-galactosidase enzymes 1, 2 and 3 (AGL1, AGL2 and AGL3), as well as the amino acid sequences of those enzymes, have been deposited in NCBI's GENBANK® database as accession numbers Z69253 (GID: 1580815), Z69254 (GID: 1580817) and Z69255 (GID: 1580811), respectively. These GENBANK® database accessions are incorporated by reference in their entirety, including the nucleic acid and protein sequences therein and the annotation of those sequences.

The amino acid sequences for over 500 different alpha-galactosidases are known and have been deposited in NCBI's GENBANK® database, including those from mammals (See e.g., accession no. CAA29232; GID: 757912), plants (See e.g., accession no. NP_{—}974447; GID: 42572703), and bacteria (See e.g., accession no. BAB38524; GID: 13364578). Further, the atomic coordinates of at least five alpha-galactosidase enzymes, including those from human, rice, and T. reesei (See e.g., Golubev et al., J. Mol. Biol., 339: 413-422 [2004]) are known. Amino acids that are conserved in alpha-galactosidase enzymes, including those from T. reesei, are also known (See e.g., Margolles-Clark et al supra; and NCBI's conserved domain accession no. COG3345.2).

In some other embodiments, the alpha-galactosidase enzyme is immunologically related to a wild-type Trichoderma reesei alpha-galactosidase, methods for the identification of which are well known in the molecular biology arts.

It is intended that the alpha-galactosidase enzymes of the present invention be produced using any suitable methods. For example, in some embodiments, the enzyme is secreted into the periplasm (e.g., by Gram-negative organisms, such as E. coli), or into the extracellular space e.g., by Gram-positive organisms, (such as Bacillus and Actinomycetes), or eukaryotic hosts (e.g., Trichoderma, Aspergillus, Saccharomyces, and Pichia).

In some embodiments, the alpha-galactosidase enzyme is produced by expressing a fusion protein containing a signal sequence operably linked to the alpha-galactosidase enzyme in a T. reesei host cell. In some of these embodiments, the alpha-galactosidase enzyme is secreted into culture medium, from which it is harvested. The signal sequence of a subject fusion protein comprises any signal sequence that facilitates protein secretion from the Trichoderma host cell. In some embodiments, the signal sequence employed is endogenous to the Trichoderma host cell, while in other embodiments, it is non-endogenous. In some other embodiments, it is a signal sequence of a protein that is known to be highly secreted from a Trichoderma sp. host cell. Such signal sequence include, but are not limited to: the signal sequence of cellobiohydrolase I, cellobiohydrolase II, endoglucanases I, endoglucanases II, endoglucanases III, alpha-amylase, aspartyl proteases, glucoamylase, mannanase, glycosidase and barley endopeptidase B (See e.g., Saarelainen, Appl. Environ. Microbiol., 63: 4938-4940 [1997]). In some embodiments, and as further described in the Examples, the alpha-galactosidase is secreted using its own signal sequence (i.e., the AGL1, AGL2 or AGL3 signal sequences, as described in Margolles-Clark et al., supra).

In some embodiments, the alpha-galactosidase is produced using a nucleic acid comprising: a signal sequence-encoding nucleic acid operably linked to an alpha-galactosidase-encoding nucleic acid, where translation of the nucleic acid produces a fusion protein comprising an alpha-galactosidase portion having an N-terminal signal sequence for secretion of the alpha-galactosidase portion from a Trichoderma host cell.

In some embodiments, the fusion protein further contains, in addition to a signal sequence, a “carrier protein” that is a portion of a protein that is endogenous to and highly secreted by the T. reesei sp. host cell. Suitable carrier proteins include, but are not limited to those of T. reesei mannanase I (Man5A, or MANI), T. reesei cellobiohydrolase II (Cel6A, or CBHII) (See e.g., Paloheimo et al., Appl. Environ. Microbiol., 69:7073-7082 [2003]) or T. reesei cellobiohydrolase I (CBHI). In some embodiments, the carrier protein is a truncated T. reesei CBH1 protein that includes the CBH1 core region and part of the CBH1 linker region. In some embodiments, the present invention comprises a nucleic acid encoding a fusion protein containing, from amino-terminus to carboxy-terminus, a signal sequence, a carrier protein and an alpha-galactosidase in operable linkage.

In some embodiments, the coding sequence of the alpha-galactosidase is codon optimized for expression of the alpha-galactosidase in the host cell used. Since codon usage tables listing the usage of each codon in many host cells, including Trichoderma reesei, are known in the art (See e.g., Nakamura et al., Nucl. Acids Res., 28: 292 [2000]) or readily derivable, such nucleic acids are readily designed to give the amino acid sequence of an alpha-galactosidase to be expressed.

In addition to a coding sequence, in some embodiments, the nucleic acid further comprises other elements that are necessary for expression of the alpha-galactosidase enzyme in the host cell. For example, in some embodiments, the nucleic acid contains a promoter for transcription of the coding sequence, and a transcriptional terminator. Exemplary promoters include, but are not limited to the T. reesei cbh1, cbh2, egl1, egl2, eg5, xln1 and xln2 promoters, or hybrids or truncated versions thereof. For example, in some embodiments, the promoter is a T. reesei cbh1 promoter. Suitable terminators include, but are not limited to the T. reesei cbh1, cbh2, egl1, egl2, eg5, xln1 and xln2 terminators, and many others, including, for example, the terminators from A. niger or A. awamori glucoamylase genes (See, Nunberg et al., [1984], supra; and Boel et al., [1984], supra), Aspergillus nidulans anthranilate synthase genes, Aspergillus oryzae TAKA amylase genes, or A. nidulans trpC (Punt et al., Gene 56:117-124 [1987]). In some embodiments, the promoter and/or terminator are native to the Trichoderma sp. host cell, while in other embodiments, they are non-endogenous.

In some embodiments, a T. reesei host cell is employed for expression of the alpha-galactosidase enzyme. In some preferred embodiments, the cell is genetically modified to reduce expression of secreted proteins that are endogenous to the cell. In some embodiments, the cell contains one or more native genes, particularly genes that encode secreted proteins, that have been deleted or inactivated. For example, in some embodiments, one or more protease-encoding genes (e.g., an aspartyl protease-encoding gene; See, Berka et al., Gene 86:153-162 [1990]; and U.S. Pat. No. 6,509,171) or cellulase-encoding genes are deleted or inactivated. In some embodiments, the Trichoderma sp. host cell is a T. reesei host cell containing inactivating deletions in the cbh1, cbh2 and egl1, and egl2 genes, as described in WO 05/001036. In some embodiments, the above-described nucleic acid is present in the nuclear genome of the Trichoderma sp. host cell, while in other embodiments, it is present in a plasmid that replicates in the Trichoderma host cell.

It is intended that the nucleic acid be introduced into the Trichoderma host cell using any one of a number of suitable techniques (e.g., electroporation, nuclear microinjection, transduction, transfection, [e.g., lipofection mediated and DEAE-Dextrin mediated transfection], incubation with calcium phosphate DNA precipitate, high velocity bombardment with DNA-coated microprojectiles, and protoplast fusion). General transformation techniques are known in the art (See e.g., WO 05/001036; U.S. Pat. No. 6,022,725; U.S. Pat. No. 6,103,490; U.S. Pat. No. 6,268,328; and published U.S. patent applications 20060041113, 20060040353, 20060040353 and 20050208623, all of which are incorporated herein by reference). In some embodiments, the preparation of Trichoderma for transformation includes the preparation of protoplasts from fungal mycelia. (See, Campbell et al., Curr. Genet. 16:53-56 [1989]). In some embodiments, the mycelia are obtained from germinated vegetative spores.

In some embodiments, once it is secreted in to culture medium, the alpha-galactosidase enzyme is recovered using any convenient method (e.g., by precipitation, centrifugation, affinity, filtration or any other method) known in the art. For example, affinity chromatography (Tilbeurgh et al., FEBS Lett., 16:215 [1984]); ion-exchange chromatographic methods (Goyal et al., Biores. Technol., 36:37 [1991]; Fliess et al., Eur. J. Appl. Microbiol. Biotechnol., 17:314 [1983]; Bhikhabhai et al., J. Appl. Biochem. 6:336 [1984]; and Ellouz et al., Chromatography 396:307 [1987]), including ion-exchange using materials with high resolution power (Medve et al., (J. Chromatography A 808:153 [1998]; hydrophobic interaction chromatography (Tomaz and Queiroz, J. Chromatography A 865:123 [1999]; two-phase partitioning (Brumbauer et al., (Bioseparation 7:287 [1999]); ethanol precipitation; reverse phase HPLC; chromatography on silica or on a cation-exchange resin (e.g., DEAE); chromatofocusing; SDS-PAGE; ammonium sulfate precipitation; or gel filtration using (e.g., Sephadex G-75), find use. In some embodiments, the alpha-galactosidase is used without purification from the other components the culture medium. In some of these embodiments, the culture medium is simply concentrated and then used without further purification of the protein from the components of the growth medium, or used without any further modification.

### Cleaning Compositions

The present invention provides cleaning compositions comprising an above-described alpha-galactosidase enzyme. In some embodiments, the cleaning composition is a fabric cleaning composition (i.e., a laundry detergent), a surface cleaning composition, a dish cleaning composition, or an automatic dishwasher detergent composition. Formulations for exemplary cleaning compositions are described in great detail in WO0001826, which is incorporated by reference herein.

In some embodiments, the subject cleaning composition (e.g., laundry or dishwashing detergent) contains from about 1% to about 80%, (e.g., about 5% to about 50%) (by weight) of at least one surfactant, (e.g., non-ionic surfactants, cationic surfactants, anionic surfactants or zwitterionic surfactants, or any mixture thereof). Exemplary surfactants include, but are not limited to alkyl benzene sulfonate (ABS), including linear alkyl benzene sulfonate and linear alkyl sodium sulfonate, alkyl phenoxy polyethoxy ethanol (e.g., nonyl phenoxy ethoxylate or nonyl phenol), diethanolamine, triethanolamine and monoethanolamine. Exemplary surfactants that may be present in detergents, particularly laundry detergents, are described in U.S. Pat. Nos. 3,664,961, 3,919,678, 4,222,905, and 4,239,659.

In some embodiments, the cleaning compositions are in solid (e.g., in powder or tablet form) or liquid form. In some additional embodiments, the cleaning compositions further comprise at least one buffer (e.g., sodium carbonate, sodium bicarbonate), detergent builder, bleach, bleach activator, enzyme, enzyme stabilizing agent, suds booster, suppresser, anti-tarnish agent, anti-corrosion agent, soil suspending agent, soil release agent, germicide, pH-adjusting agent, non-builder alkalinity source, chelating agent, organic or inorganic filler, solvent, hydrotrope, optical brightener, dye, perfume, etc. In some embodiments, the cleaning composition is combined with a detergent before use as a laundry additive.

In some embodiments, the subject cleaning composition contains a further non-starch food polysaccharide degrading enzyme (e.g., hemicellulase, mannanase, pectinase, xylanase, or pectate lyase) and, optionally, one or more additional enzyme such as a protease such as a subtilisin protease and/or SSI protein, lipase, amylase, cellulase, cutinase, lipase, oxidoreductase, etc., for the removal of other stains.

A wide variety of other ingredients useful in detergent cleaning compositions also find use in the compositions provided herein, including other active ingredients, carriers, hydrotropes, processing aids, dyes or pigments, solvents for liquid formulations, etc. In some embodiments in which additional sudsing is desired, suds boosters such as the C₁₀-C₁₆ alkolamides are incorporated into the compositions, typically at about 1% to about 10% levels.

In some embodiments, the detergent compositions comprise water and/or other solvents as carriers. Low molecular weight primary or secondary alcohols exemplified by methanol, ethanol, propanol, and isopropanol are suitable for use. Monohydric alcohols are preferred for solubilizing surfactants, but polyols such as those containing from about 2 to about 6 carbon atoms and from about 2 to about 6 hydroxy groups (e.g., 1,3-propanediol, ethylene glycol, glycerine, and 1,2-propanediol) also find use. In some embodiments, the compositions comprise from about 5% to about 90% (typically from about 10% to about 50%) of such carriers.

In some embodiments, the detergent compositions herein are formulated such that during use in aqueous cleaning operations, the wash water has a pH between about 5.0 and about 11.5. Thus, finished products are typically formulated at this range. Techniques for controlling the pH at recommended usage levels include the use of buffers, alkalis, acids, etc., and are well known to those skilled in the art. In some embodiments, the cleaning composition is an automatic dishwashing detergent that has a working pH in the range of about pH 9.0 to about pH 11.5, about pH 9.0 to about pH 9.5, about pH 9.5 to about pH 10.0, about pH 10.0 to about pH 10.5, about pH 10.5 to about pH 11.0, or about pH 11.0 to about pH 11.5. In some other embodiments, the cleaning composition is a liquid laundry detergent that has a working pH in the range of about pH 7.5 to about pH 8.5, about pH 7.5 to about pH 8.0, or about pH 8.0 to about pH 8.5. In some other embodiments, the cleaning composition is a solid laundry detergent that has a working pH in the range of about pH 9.5 to about pH 10.5, about pH 9.5 to about pH 10.0, or about pH 10.0 to about pH 10.5.

The cleaning compositions described herein require an effective amount of the alpha-galactosidase. In some embodiments, he working concentration of the subject alpha-galactosidase enzyme in the cleaning composition is about 0.01 ppm (parts per million, w/v) to about 100 ppm, about 0.01 ppm to about 0.05 ppm, about 0.05 ppm to about 0.1 ppm, about 0.1 ppm to about 0.5 ppm, about 0.5 ppm to about 1 ppm, about 1 ppm to about 5 ppm, about 5 ppm to about 10 ppm, or about 10 ppm to about 100 ppm.

Various bleaching compounds, such as the percarbonates, perborates and the like, also find use in the cleaning compositions of the present invention. In some embodiments, these are typically present at levels from about 1% to about 15% by weight. In some additional embodiments, such compositions also contain bleach activators (e.g., tetraacetyl ethylenediamine, nonanoyloxybenzene sulfonate, and the like), known in the art. Usage levels typically range from about 1% to about 10% by weight.

Various soil release agents, especially of the anionic oligoester type, various chelating agents, especially the aminophosphonates and ethylenediaminedisuccinates, various clay soil removal agents, especially ethoxylated tetraethylene pentamine, various dispersing agents, especially polyacrylates and polyasparatates, various brighteners, especially anionic brighteners, various suds suppressors, especially silicones and secondary alcohols, various fabric softeners, especially smectite clays, and the like also find use in the present compositions at levels ranging from about 1% to about 35% by weight. Standard formularies are well-known to those in the art.

Enzyme stabilizers also find use in the cleaning compositions of the present invention. Such stabilizers include, but are not limited to propylene glycol (preferably from about 1% to about 10%), sodium formate (preferably from about 0.1% to about 1%), and calcium formate (preferably from about 0.1% to about 1%).

In some embodiments, hard surface cleaning compositions and fabric cleaning compositions further comprise various builders at levels from about 5% to about 50% by weight. Typical builders include the 1-10 micron zeolites, polycarboxylates such as citrate and oxydisuccinates, layered silicates, phosphates, and the like. Other conventional builders are listed in standard formularies.

Other optional ingredients include chelating agents, clay soil removal/anti redeposition agents, polymeric dispersing agents, bleaches, brighteners, suds suppressors, solvents and aesthetic agents.

The present cleaning compositions find use in suitable cleaning methods. In some embodiments, the cleaning methods include: contacting an isolated alpha-galactosidase enzyme comprising an amino acid sequence that is related to an alpha-galactosidase of Trichoderma reesei with an object (e.g., a fabric or dish) under conditions suitable for activity of said alpha-galactosidase enzyme, to clean the object. Depending on the working pH of the cleaning composition employed, the alpha-galactosidase enzyme is contacted with the object at a pH of, for example, about pH 5 to about 6.5, about pH 6.5 to about 7.5, about pH 7.5 to about 8.5, about pH 9.5 to about 10.5, or about pH 10.0 to about 11.5. In some embodiments, the object is a soiled object and in some further embodiments, the object is stained with a foodstuff containing a non-starch food polysaccharide such as a galactomannan gum (e.g., guar gum or lima beam gum, etc.). In some further embodiments, the object is stained with chocolate cream, ice cream or salad dressing.

The cleaning composition described herein is more effective at removal of certain stains (e.g., stains from foodstuffs containing galactomannan polysaccharides), than equivalent cleaning compositions that do not contain an alpha-galactosidase. In some embodiments, the cleaning compositions of the present invention is more effective at stain removal in comparison to an otherwise equivalent cleaning composition that does not contain alpha-galactosidase enzyme. Using a standard reflectometer-based assay such as that described in Example 4, for example, some cleaning compositions of the present invention remove and/or discolor at least about 20%, at least about 40%, at least about 60%, at least about 80% or, at least about 90% more stain than an equivalent cleaning composition that does not contain the alpha-galactosidase.

### EXPERIMENTAL

The following Examples are provide those of ordinary skill in the art with a complete disclosure and description of how to make and use the present invention, and are not intended to limit the scope of the invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Cloning of Alpha-Galactosidase Genes

Coding sequences from the agl1, agl2, agl3 and man1 genes of Trichoderma reesei strain QM6A were amplified by PCR using the following primers:

| Primer | | | SEQ ID |
|---|---|---|---|
| name | Sequence | Notes | NO |
| NSP061 | GGGGACAAGTTTGTACAAAAAAGCAGGCT | Gateway | SEQ ID |
| | ATGACCCCTCACTCGATTGACC | AGL1 | NO: 1 |
| | | forward | |
| NSP062 | GGGGACCACTTTGTACAAGAAAGCTGGGT | Gateway | SEQ ID |
| | TCACCAGTTTCGGCACTTCTTGC | AGL1 | NO: 2 |
| | | reverse | |
| NSP081 | GGGGACAAGTTTGTACAAAAAAGCAGGCT | Gateway | SEQ ID |
| | ATGCTCGGCGCTCCCTCTCC | AGL2 | NO: 3 |
| | | forward | |
| NSP082 | GGGGACCACTTTGTACAAGAAAGCTGGGT | Gateway | SEQ ID |
| | TCATGTCTGCTTCTCCAAAAACACC | AGL2 | NO: 4 |
| | | reverse | |
| NSP091 | GGGGACAAGTTTGTACAAAAAAGCAGGCT | Gateway | SEQ ID |
| | ATGTCGCCCAGTGCTGCAGTTC | AGL3 | NO: 5 |
| | | forward | |
| NSP092 | GGGGACCACTTTGTACAAGAAAGCTGGGT | Gateway | SEQ ID |
| | CTAGTGAGTCCTTTTCAGGCGC | AGL3 | NO: 6 |
| | | reverse | |
| NSP201 | GGGGACAAGTTTGTACAAAAAAGCAGGCT | Gateway | SEQ ID |
| | ATGATGATGCTCTCAAAGAGTCTCC | MAN1 | NO: 7 |
| | | forward | |
| NSP202 | GGGGACCACTTTGTACAAGAAAGCTGGGT | Gateway | SEQ ID |
| | TCATGTATTCAGGCATTGCGAGTACC | MAN1 | NO: 8 |
| | | reverse | |

and cloned into the pTREX3g vector using the GATEWAY™ recombination system (Invitrogen Corporation, Carlsbad, Calif.). pTREX3g is described in detail in Example 6 of WO05/001036.

### Example 2

### Transformation of T. reesei Cells

All vectors were transferred into the quad deleted (Δchb1, Δcbh2, Δegl1, and Δegl2) T. reesei strain (WO 05/001036) originally derived from RL-P37 (Sheir-Neiss et al., Appl. Microbiol. Biotechnol., 20:46-53 [1984]; U.S. Pat. No. 4,797,361) or a 1A52pyr4⁻ strain, by particle bombardment.

A suspension of spores (approximately 5×10⁸ spores/ml) from the Trichoderma strain to be transformed was prepared. 100 ul-200 ul of spore suspension was spread onto the center of plates of MM acetamide medium (MM acetamide medium has the following composition: 0.6 g/L acetamide; 1.68 g/L CsCl; 20 g/L glucose; 20 g/L KH₂PO₄; 0.6 g/L CaCl₂.2H₂O; 1 ml/L 1000× trace elements solution; 20 g/L Noble agar; pH 5.5. 1000× trace elements solution contained 5.0 g/l FeSO₄.7H₂O, 1.6 g/l MnSO₄.H₂O, 1.4 g/l ZnSO₄.7H₂O and 1.0 g/l CoCl₂.6H₂O). The spore suspension was allowed to dry on the surface of the MM acetamide medium.

Biolistic transformation of Trichoderma cells was accomplished using a Biolistic® PDS-1000/he Particle Delivery System from Bio-Rad (Hercules, Calif.) following the manufacturer's instructions (See e.g., WO 05/001036 and US Pat. Publ. No. 2006/0003408).

### Example 3

### Enzyme Activity Analysis

Cultures of cells containing vectors for agl1, agl2, agl3 and man1 were grown in a culture flask and supernatant from each of the cultures was analyzed using SDS PAGE. For each culture supernatant, alpha-galactosidase activity was measured in Mcllvaine buffer using 4-nitrophenyl-alpha-D-galactopyranoside as a substrate. Enzyme activity assays were performed using the Sigma protocol (Enzymatic Assay of Alpha-Galactosidase, Sigma Product Information; See also, McCleary, Meth. Enzymol., 160:627-632 [1988]; and alpha-galactosidase technical data sheets from A. niger and Guar Seed, Megazyme), as briefly described below.

First, 0.10 ml of substrate was added to 16×125 mm glass tubes, which were then heated in a water bath to temperature by incubating at least 5′ to the desired temperature. Then, 0.10 ml of diluted enzyme was added to each tube at 15 second intervals and vortexed to mix the enzyme with substrate. The mixtures were incubated for 5′ at prescribed temperatures for testing (30° C., 37° C., 40° C., 45° C., 60° C., and 75° C.). To stop the reaction, 3.0 ml of 2% sodium carbonate was added at the same 15 second intervals. The solutions were mixed and the tubes removed from the water bath for reading at 410 nm.

In these experiments, the enzyme dilutions were prepared to 10 mM in the appropriate buffer at each pH (McIlvaine with a pH of 2.1, 2.5, 3, 4, 5, 6, 7; 80.1M sodium acetate at pH 4.5). The enzyme substrate for primary testing was 4-nitrophenyl-alpha-D-galactopyranoside (Sigma, cat: N0877, MW: 301.25). Blanks were included with each test, including substrate, stop reagent, and enzyme blanks (p-nitrophenol was used as a substrate reference).

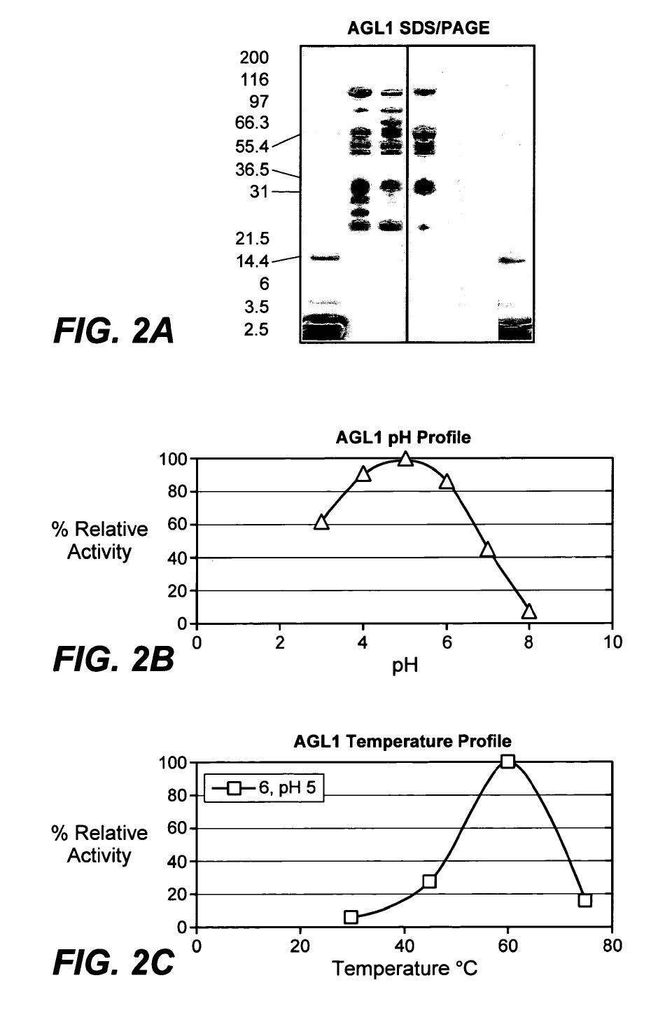

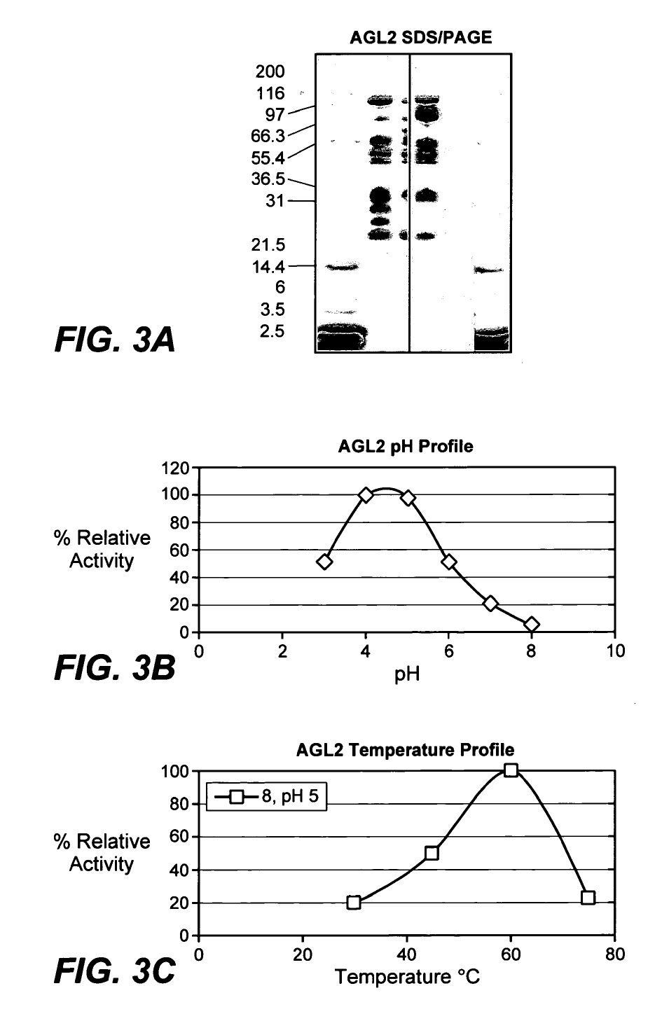

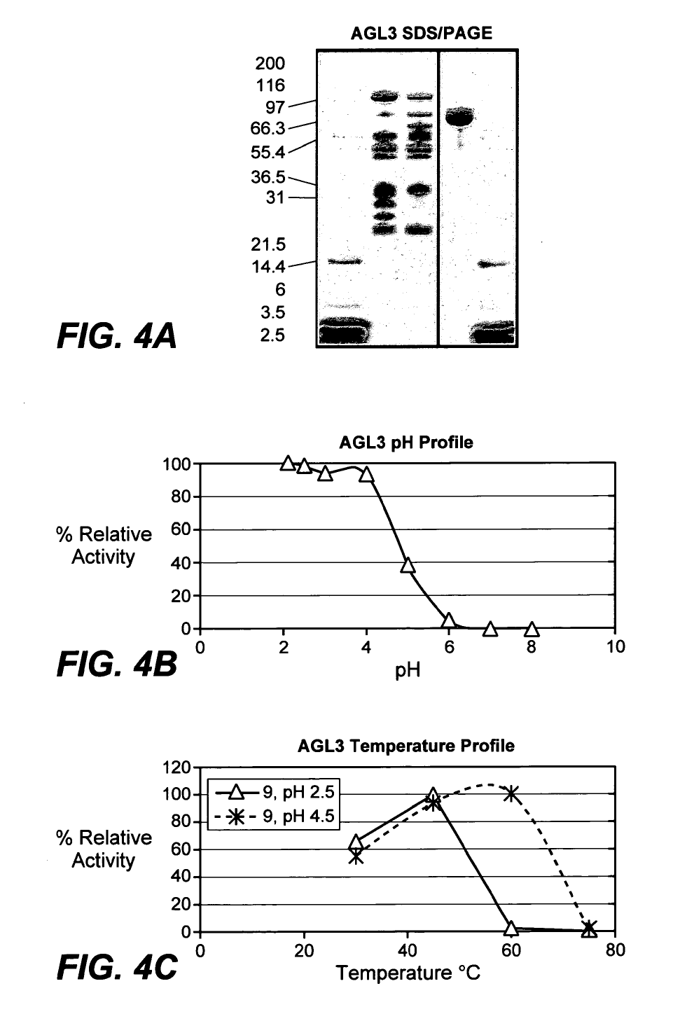

The SDS-PAGE gels shown in FIGS. 2-4 show that for each supernatant, a protein of approximately the correct size was produced. Under the assay conditions used, AGL1 (which has a predicted molecular weight of 45.7 kDa) had a pH optimum of pH 5 and a temperature optimum of about 60° C. (FIG. 2), AGL2 (which has a predicted molecular weight of 79.5 kDa) had a pH optimum of pH 4-5 and a temperature optimum of about 60° C. (FIG. 3) and AGL3 (which has a predicted molecular weight of 66.3 kDa) had a pH optimum of pH 2-4, a temperature optimum of about 60° C. at pH 4.5 and a temperature optimum of 45° C. at pH 2.5 (FIG. 4).

### Example 4

### Disk Assay Analysis of Cleaning Activity of Trichoderma Alpha-Galactosidase Proteins

AGL1, AGL2 and AGL3 were tested for their ability to clean swatches stained with chocolate cream, salad dressing, and guar-pigment using the following method.

Salad dressing with pigment (STC CFT CS-6), chocolate cream (STC EMPA 160) and guar-pigment (STC CFT CS-43) were soiled cotton swatches (Test Fabrics, Inc. West Pittston, Pa., USA). Chocolate ice cream circles (4 cm soil on 10 cm cotton swatches) were obtained from Warwick-Equest Limited, Consett, County Durham, England.

Swatches for the microplate assay were cut into 15 cm circles (disks) with textile Punch Press Model B equipped with a ⅝″ die cutter. Single disks were placed into each well of a 24-well microplate (Costar 3526). One (1) ml of washing solution containing per liter, 1.5 ml AATCC HDL (standard liquid detergent) detergent, 50 mM Hepes buffer pH 7.4 was added to each well. 1 to 20 ug of diluted enzyme was added with a positive displacement pipette. The AATCC 2003 Standard Liquid Detergent contained 12% linear alkyl sulfonates, 8% alcohol ethoxylates, 8% propanediol, 1.2% citric acid, 4% fatty acid and 4% sodium hydroxide with the balance being water. Control wells contained no enzyme. The microplate was covered with its plastic lid and incubated at 37 C with 100 rpm gentle rotation. After 4-16 hr, the supernatants were removed by aspiration and each well was washed three times with 1.5 ml of Dulbecco's PBS pH 7.3 and three times with 1.5 ml of distilled water. Each disk was removed from its well and dried overnight in air. Disks were inspected visually and analyzed with a Minolta Reflectometer CR-200 calibrated on a standard white tile. The average L values were calculated with the percent standard deviation of the data with generally 4 replicates per control and test sample.

Experiments using Heavy Duty Detergent (HDD) or Automatic Dish Washing (ADW) detergent used 0.015% to 0.1% AATCC HDD without phosphate pH 10, and 0.015% to 0.1% WFK ADW detergent Type B without phosphate were performed. AATCC 1993 standard reference heavy duty detergent without brightner contained 18% linear alkyl sulfonates, 2% linear fatty alcohol ethoxylate, and sodium carbonate to 100%. 25% Zeolite A, 18% soda ash, 0.5% sodium silicates, 22.13% sodium sulfate, 10% moisture and space (6.28%) for copolymers, enzymes, or carboxy methyl cellulose. WFK automatic dishwashing detergent Type B without brightner and without phosphate contained 30% sodium citrate dehydrate, 12% maleic acid sodium salt, sodium perborate monohydrate, 2% tetraacetyl ethylenediamine, 25% sodium disilicate, 2% linear fatty alcohol ethoxylate, and anhydrous sodium carbonate to 100%.

In the following examples and accompanying figures, the protein extract containing AGL1 is referred to as NSP-6, the protein extract containing AGL2 is referred to as NSP-8, and the protein extract containing AGL3 is referred to as NSP-9.

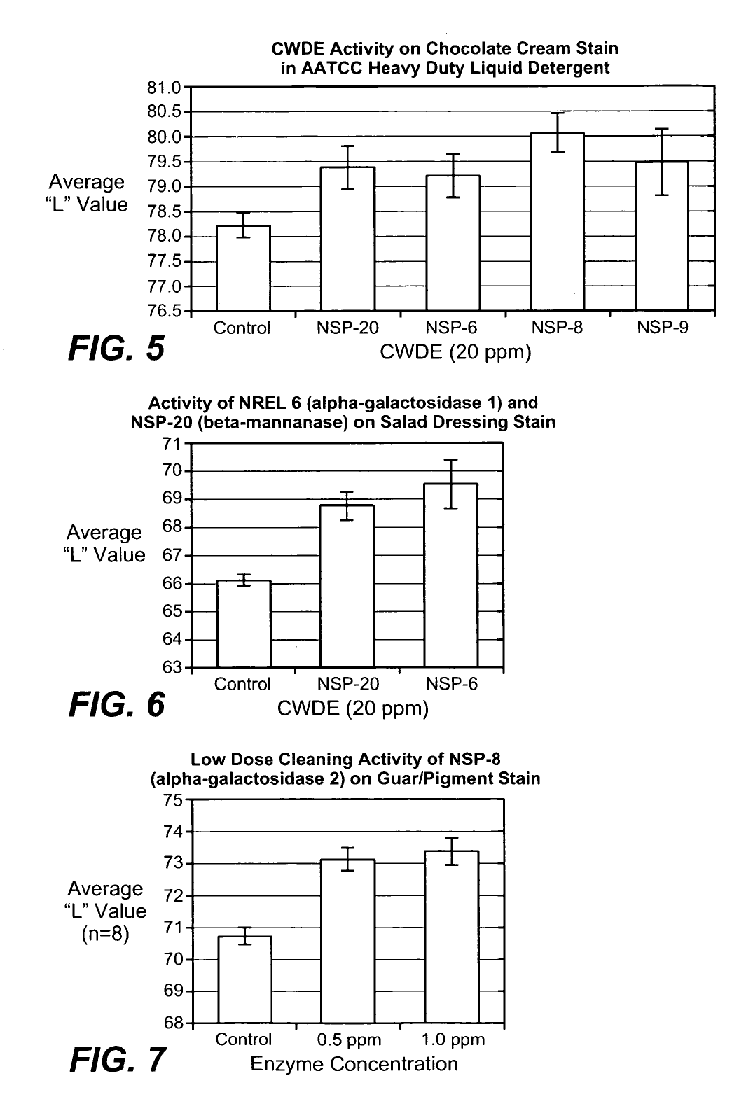

As shown in FIG. 5, NSP-6 (alpha-galactosidase 1), NSP-8 (alpha-galactosidase 2), and NSP-9 (alpha-galactosidase 3), showed excellent cleaning on chocolate cream stain in 0.15% AATCC heavy duty liquid detergent using the microplate disk method. NSP-20, a beta-. CWDE is an abbreviation for cell-wall degrading enzymes. FIG. 6 shows alpha-galactosidase 1 cleaning on salad dressing stain in 0.022% AATCC heavy duty liquid detergent pH 7.4. FIG. 7 shows that low concentrations (0.5 to 1.0 ppm) of NSP-8 (alpha-galactosidase 2) give significant cleaning on guar-pigment technical stain in 0.15% AATCC heavy duty liquid detergent.

### Example 5

### Tergotometer Analysis of Cleaning Activity of Trichoderma Alpha-Galactosidase Proteins

Tergotometer studies used a 6 pot Tergotometer Model 7243S (U.S. Testing, Co. Inc. Hoboken, N.J.) maintained at 37 C. Agitation speed was set to 100 rpm. Six chocolate ice cream circles on cotton swatches were added to 1 liter of AATCC HDL detergent containing 6 gpg hardness (diluted from stock 15000 gpg hardness solution containing 1.735 M calcium chloride and 0.67 M magnesium chloride) and 50 mM Hepes buffer pH 7.4.

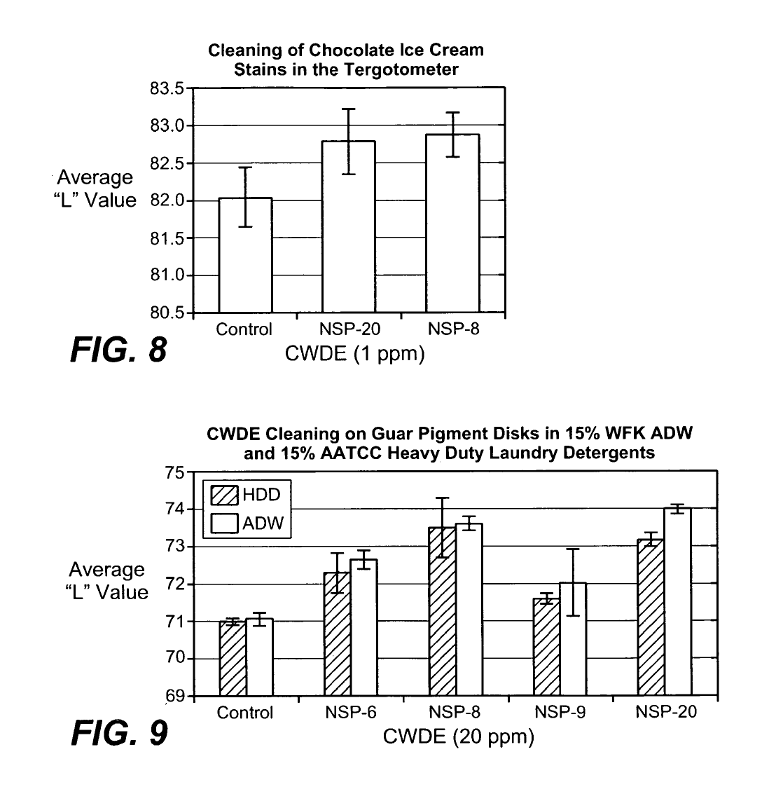

FIG. 8 shows that alpha-galactosidase 2 (NSP-8) cleans ice cream swatches under tergotometer conditions with 1 ppm of enzyme in 30 min.

FIG. 9 shows that all three alpha-galactosidases and especially alpha-galactosidase 2 (NSP-8) showed significant cleaning in the microplate disk method (as described in Example when dosed at 20 ppm in 0.015% automatic dishwashing detergent (WFK) pH 10.5, or in 0.015% AATCC solid laundry detergent, pH 10.2.

The above examples demonstrate that Trichoderma reesei alpha-galactosidases enzymes effectively remove soil from cotton swatches stained with salad dressing, chocolate cream, ice cream and guar-pigment stains. The activity on guar technical stains may be the basis of the cleaning as salad dressing and ice creams often contain guar gum as an ingredient. The alpha-galactosidase enzymes tested perform well in pH ranges well beyond that expected.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as should not be unduly limited to such specific embodiments. Indeed, various modifications of the described modes for carrying out the invention that are obvious to those skilled in the art and/or related fields are intended to be within the scope of the present invention.

Having described exemplary embodiments of the present invention, it will appear to those ordinarily skilled in the art that various modifications may be made to the disclosed embodiments, and that such modifications are intended to be within the scope of the present invention.

Those of skill in the art readily appreciate that the present invention is well adapted to carry out the objects and obtain the ends and advantages mentioned, as well as those inherent therein. The compositions and methods described herein are representative and are not intended as limitations on the scope of the invention. It is readily apparent to one skilled in the art that varying substitutions and modifications may be made to the invention disclosed herein without departing from the scope and spirit of the invention.

The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

What is claimed is:

1. A cleaning composition comprising an isolated alpha-galactosidase enzyme comprising an amino acid sequence that is at least about 90% identical to an alpha-galactosidase of Trichoderma reesei.

2. The cleaning composition of claim 1, further comprising at least one surfactant.

3. The cleaning composition of claim 1, wherein said cleaning composition has a working pH that is greater than about pH 5.

4. The cleaning composition of claim 1, wherein said cleaning composition is a solid.

5. The cleaning composition of claim 1, wherein said cleaning composition is a liquid.

6. The cleaning composition of claim 1, wherein said cleaning composition comprises a laundry detergent.

7. The cleaning composition of claim 1, wherein said cleaning composition comprises a dishwashing detergent.

8. The cleaning composition of claim 1, further comprising one or more additional enzymes.

9. The cleaning composition of claim 8, wherein said additional enzyme is selected from hemicellulases, mannanases, pectinases, amylases, xylanases, pectin lyases, proteases, cellulases, cutinases, lipases, and oidoreductases

10. The cleaning composition of claim 1, wherein said isolated alpha-galactosidase enzyme is immunologically cross-reactive with an alpha-galactosidase of Trichoderma reesei.

11. A cleaning method comprising:
contacting an isolated alpha-galactosidase enzyme comprising an amino acid sequence that is at least about identical to an alpha-galactosidase of Trichoderma reesei, with an object under conditions suitable for activity of said alpha-galactosidase enzyme, to clean said object.

12. The method of claim 11, wherein said alpha-galactosidase is contacted with said object at a pH that is greater than about pH 5.

13. The method of claim 11, wherein said object is a soiled object.

14. The method of claim 13, wherein said soiled object comprises a non-starch food polysaccharide.

15. The method of claim 14, wherein said non-starch food polysaccharide is a galactomannan gum.

16. The method of claim 14, wherein said non-starch food polysaccharide is guar gum or lima beam gum.

17. The method of claim 13, wherein said soiled object is stained with chocolate cream, ice cream or salad dressing.

18. The method of claim 11, wherein said object is fabric.

19. The method of claim 11, wherein said object is dishware.
